(19) United States
(12) Patent Application Publication
(10) Pub. No.: US 2011/0160820 A1
(43) Pub. Date: Jun. 30, 2011

(54) VARIABLE STIFFNESS MULTI-LUMEN LEAD BODY FOR AN IMPANTABLE LEAD

(76) Inventors: Timothy R. Jackson, Minneapolis, MN (US); Patrick J. Willoughby, Hugo, MN (US)

(21) Appl. No.: 12/939,884

(22) Filed: Nov. 4, 2010

Related U.S. Application Data
(60) Provisional application No. 61/291,219, filed on Dec. 30, 2009.

(51) Int. Cl. A61N 1/05; B29C 45/14

(52) U.S. Cl. 607/116; 264/266

(57) ABSTRACT

A multi-lumen lead body having a smooth transition between regions of varying stiffness on the lead body is described. In one example, the different regions of the lead body can be formed by altering a mix ratio of the different polymers used to form the lead body during the extrusion process. In another example, two or more layers of polymeric materials are co-extruded, and the cross-sectional thicknesses of each of the layers varied in the different regions of the lead body to achieve different stiffnesses. In yet another example, the internal geometry of the lead body is altered during the extrusion process to alter the physical properties of the lead body in a select region of the lead body. In still yet another example, a transition member is provided between regions of varying stiffness to facilitate a smooth transition between the different regions.

### CROSS REFERENCE TO RELATED APPLICATIONS

This application claims the benefit under 35 U.S.C. §119 of U.S. Provisional Application No. 61/291,219, filed on Dec. 30, 2009, entitled “Variable Stiffness Multi-Lumen Lead Body for an Implantable Lead,” which is incorporated herein by reference in its entirety for all purposes.

### TECHNICAL FIELD

The present invention relates to implantable medical electrical leads and more particularly, to a variable stiffness, multi-lumen lead body having a smooth transition between regions of varying stiffness.

### BACKGROUND

Various types of medical electrical leads for use in cardiac rhythm management (CRM) and neurostimulation systems are known. For CRM systems, such leads are typically extended intravascularly to an implantation location within or on a patient's heart, and are thereafter coupled to a pulse generator or other implantable device for sensing cardiac electrical activity, delivering therapeutic stimuli, and the like. The leads are desirably highly flexible to accommodate natural patient movement, yet also constructed to have minimized profiles.

### SUMMARY

In Example 1, a medical electrical lead comprises a lead body having a proximal end, a distal end and at least two lumens extending within the lead body. The lead body includes a proximal portion comprising a first material, a middle portion comprising a second material having a lower durometer than the first material, and a distal portion comprising a third material having a lower durometer than the second material. At least one conductor extends within one of the two or more lumens. The lead also comprises at least one electrode located on the distal portion of the lead body and operatively connected to the at least one conductor.

In Example 2, the lead according to Example 1, wherein the proximal and middle portions each comprise a polyurethane material and the distal portion comprises a silicone material.

In Example 3, the lead according to any one of Examples 1-2, wherein the proximal portion and middle portion each comprise a mixture of at least two polyurethane materials, wherein each polyurethane material has a different durometer.

In Example 4, the lead according to any one of Examples 1-3, wherein the proximal portion includes a first ratio of the at least two polyurethanes and the middle portion includes a second ratio of the at least two polyurethanes.

In Example 5, the lead according to any one of Examples 1-4, wherein the proximal portion and middle portion each include a first material layer having a first stiffness and a second material layer having a second stiffness, wherein the thickness of at least one of the layers is varied to reduce the stiffness of the middle portion relative to the proximal portion.

In Example 6, the lead according to any one of Examples 1-5, wherein the distal portion comprises a silicone material coupled to the middle portion at a transition region.

In Example 7, the lead according to any of Examples 1-6, wherein the transition region includes a transition element coupled to the middle portion and the distal portion.

In Example 8, the lead according to any one of Examples 1-7, wherein the transition element is a tubular member disposed over the middle portion and under the distal portion at the transition region.

In Example 9, the lead according to any one of Examples 1-8, wherein the transition element comprises a plurality of recesses, and wherein the distal portion is molded over the transition element.

In Example 10, the lead according to any one of Examples 1-9, wherein the frequency of the recesses increases from a proximal end to a distal end of the transition element.

In Example 11, the lead according to any one of Examples 1-10, wherein a wall thickness of the proximal portion, middle portion or both is decreased from a proximal end of the lead body toward the distal portion.

In Example 12, a medical electrical lead comprises a lead body having a proximal end, a distal end and at least two lumens extending within the lead body. The lead body comprises a proximal portion comprising a first material and a distal portion comprising a second material coupled to the proximal portion at a transition region, wherein a wall thickness of the proximal portion decreases in a direction from the proximal end toward the distal portion. The lead further comprises at least one conductor extending within one of the two or more lumens, and at least one electrode located on the distal portion of the lead body and operatively connected to the at least one conductor.

In Example 13, the lead according to Example 12, wherein an outer diameter of the proximal portion is reduced to reduce the stiffness of the proximal portion in a direction towards the distal portion.

In Example 14, the lead according to any one of Examples 12-13, wherein an inner geometry of at least one of the lumens is varied to reduce the stiffness of the proximal portion in a direction towards the distal portion.

In Example 15, the lead according to any one of Examples 12-14, wherein the transition region includes a tubular transition element disposed over the proximal portion and under the distal portion at the transition region.

In Example 16, the lead according to any one of Examples 12-15, wherein the transition element comprises a plurality of recesses, and wherein the distal portion is molded over the transition element.

In Example 17, a method of fabricating a multi-lumen lead body for a medical electrical lead including at least one portion having a varying stiffness comprises the steps of: mixing a first material having a first durometer with a second material having a second durometer to form a mixture having a mix ratio of the first material to second material; and simultaneously extruding the mixture to form at least one portion of the lead body while altering the mix ratio to vary the stiffness of the at least one portion.

In Example 18, a method of fabricating a multi-lumen lead body for a medical electrical lead including at least one portion having a varying stiffness comprises the steps of: extruding at least a first material layer having a first durometer, and altering a wall thickness of the first material layer to vary the stiffness of the at least one portion of the lead body.

In Example 19, the method according to Example 18, further comprising extruding a second material layer having a second durometer and altering a wall thickness of the second material layer to vary the stiffness of the at least one portion of the lead body.

In Example 20, the method according to any of Examples 18-19, further comprising maintaining an outer diameter of the lead body.

In Example 21, the method according to any one of Examples 18-20, further comprising reducing an outer diameter of the lead body.

In Example 22, the method according to any one of Examples 18-21, further comprising changing a material composition of the first material layer from a first polymeric material to a second polymeric material.

In Example 23, the method according to any one of Examples 18-22, further comprising changing a material composition of the second material layer from a first polymeric material to a second polymeric material.

In Example 24, a method of fabricating a multi-lumen lead body for a medical electrical lead including at least one portion having a varying stiffness comprises the steps of: extruding a portion of a multi-lumen lead body including two or more lumens; and altering an inner geometry of at least one lumen to vary the stiffness of the portion of the lead body.

In Example 25, the method according to Example 24, wherein the step of altering an inner geometry of the at least one lumen comprises increasing an inner diameter of the at least one lumen.

In Example 26, the method according to any one of Examples 24-25, further comprising maintaining an outer diameter of the lead body.

In Example 27, a medical electrical lead includes a multi-lumen lead body having a proximal end, a distal end, and at least two lumens extending within the lead body. The lead body includes a first portion comprising a first polymeric material blend and a second portion located distal to the first portion comprising a second polymeric material blend having a lower durometer than the first polymeric material blend. At least one conductor extends within one of the two or more lumens. The lead also comprises at least one electrode located on the distal portion of the lead body and operatively connected to the at least one conductor.

In Example 28, the lead according to Example 27, wherein the first portion of the lead body comprises a blend of at least two polyurethane materials, each polyurethane material having a different durometer, and wherein the second portion comprises silicone.

In Example 29, the lead according to any one of Examples 27-28, wherein a ratio of the at least two polyurethanes changes along a longitudinal axis of the lead body in a direction from the proximal end towards the distal end of the lead body such that a stiffness of the lead body decreases in the same direction.

In Example 30, the lead according to any one of Examples 27-29, wherein the first portion of the lead body includes a first material layer having a first durometer and a second material layer having a second durometer, wherein the thickness of at least one of the layers is varied to vary a stiffness of the lead body such that the stiffness decreases along a longitudinal axis of the lead body in a direction from the proximal end towards the distal end of the lead body.

In Example 31, the lead according to any one of Examples 27-30, further comprising a tubular transition element disposed between and coupled to the first portion and the second portion, and wherein the second portion comprises silicone.

In Example 32, the lead according to Example 31, wherein the tubular transition element comprises a plurality of recesses, and wherein the second portion is molded over the transition element.

In Example 33, the lead according to any one of Examples 27-32, wherein a wall thickness of the first portion, second portion or both portions is decreased in a direction from a proximal end of the lead body toward the distal portion.

Example 34 is a method of fabricating a multi-lumen lead body for a medical electrical lead including at least one portion having a varying durometer. The method comprises the steps of mixing a first polymeric material having a first durometer with a second polymeric material having a second durometer to form a mixture having a mix ratio of the first polymeric material to the second polymeric material; and simultaneously extruding the mixture to form at least one portion of the multi-lumen lead body while altering the mix ratio to vary the stiffness of the at least one portion.

In Example 35, the method according to Example 34, wherein the mix ratio is altered such that the stiffness of the at least one portion is gradually and continuously reduced.

Example 36 is a method of fabricating a multi-lumen lead body for a medical electrical lead including at least one portion having a varying stiffness comprising the steps of extruding at least a first polymeric material having a first durometer and altering a wall thickness of the first material to vary the stiffness of the at least one portion of the lead body.

In Example 37, the method according to Example 36, further comprising extruding a second polymeric material having a second durometer over the first polymeric material and altering a wall thickness of the second material to vary the stiffness of the at least one portion of the lead body.

In Example 38, the method according to any one of Examples 35-36, further comprising maintaining an outer diameter of the lead body.

In Example 39, the method according to any one of Examples 35-38, further comprising reducing an outer diameter of the lead body.

In Example 40, the method according to any one of Examples 35-39, further comprising the step of mixing the first polymeric material with a second polymeric material having a second durometer to form a polymeric material blend having a mix ratio of the first material to the second material and altering the mix ratio of the first material to the second material to reduce the stiffness of the at least one portion during extrusion.

In Example 41, the method according to any one of Examples 35-40, further comprising the step of altering an inner geometry of at least one lumen to vary the stiffness of the at least one portion of the lead body.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1A is a perspective view of a medical electrical lead according to one embodiment of the present invention.

FIGS. 1B-1D are end-cross-sectional views of a lead body according to various embodiments of the present invention.

FIG. 2 is a perspective view of a medical electrical lead according to another embodiment of the present invention.

FIG. 3 is a longitudinal cross-sectional view of a portion of a lead body according to another embodiment of the present invention.

FIG. 4 is a longitudinal cross-sectional view of a portion of a lead body according to yet another embodiment of the present invention.

FIG. 5 is a longitudinal cross-sectional view of a portion of a lead body according to still another embodiment of the present invention.

FIG. 6 is a longitudinal cross-sectional view of a portion of a lead body according to still yet another embodiment of the present invention.

FIG. 7 is a schematic view of a transition member according to an embodiment of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

The leads according to the various embodiments of the present invention are suitable for sensing intrinsic electrical activity and/or applying therapeutic electrical stimuli to a patient. Exemplary applications include, without limitation, cardiac rhythm management (CRM) systems and neurostimulation systems. For example, in exemplary CRM systems utilizing pacemakers, implantable cardiac defibrillators, and/or cardiac resynchronization therapy (CRT) devices, the medical electrical leads according to the various embodiments of the invention can be endocardial leads configured to be partially implanted within one or more chambers of the heart so as to sense electrical activity of the heart and apply a therapeutic electrical stimulus to the cardiac tissue within the heart. Additionally, the leads formed according to the various embodiments of the present invention may be suitable for placement in a coronary vein adjacent to the left side of the heart so as to facilitate bi-ventricular pacing in a CRT or CRT-D system. Still additionally, leads formed according to embodiments of the present invention may be configured to be delivered intravascularly to deliver an electrical stimulation therapy to a nerve or other neurostimulation target. The medical electrical leads may be unipolar, bipolar, or multi-polar depending upon the type of therapy to be delivered.

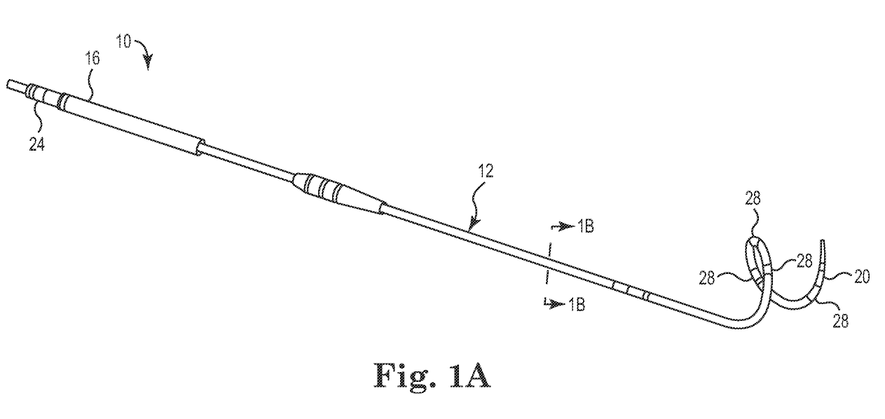

FIG. 1A is a perspective view of a medical electrical lead 10 according to various embodiments of the present invention. According to some embodiments, the medical electrical lead 10 can be configured for implantation within a patient's heart, and more particularly, within a patient's neurovascular regions. The medical electrical lead 10 includes an elongated, polymeric lead body 12 extending from a proximal end 16 to a distal end 20. In one embodiment, the distal end 20 has a tapered profile. The proximal end 16 of the lead body 12 is configured to be operatively connected to a pulse generator via a connector 24. At least one conductor (not shown) extends from the connector 24 through the lead body 12 to one or more electrodes 28 at the distal end 20 of the lead 10. The conductor can include coiled conductors, cable conductors or combinations thereof. In one embodiment, the lead 10 is a quadri-polar lead including one coiled conductor and three cable conductors. The coiled conductors can have either a co-radial or a co-axial configuration. In embodiments of the present invention employing multiple electrodes 28 and multiple conductors, each conductor is connected to an individual electrode 28 in a one-to-one manner allowing each electrode 28 to be individually addressable.

The electrodes 28 can have any electrode configuration as is known in the art. According to one embodiment of the present invention, at least one electrode can be a ring or partial ring electrode. According to another embodiment, at least one electrode 28 is a shocking coil. In some embodiments, a combination of electrode configurations may be used. The electrodes 28 can be coated with or formed from platinum, stainless steel, MP35N, a platinum-iridium alloy, or another similar conductive material. In further embodiments, a steroid eluting collar may be located adjacent to at least one electrode 28.

According to various embodiments, the lead body 12 can include one or more fixation members for securing and stabilizing the lead body 12 including the one or more electrodes 28 at a target site within a patient's body. The fixation member(s) can be active or passive. Examples of passive fixation include pre-formed distal portions of the lead body 12 such as, for example, a spiral 36, adapted to bear against the vessel walls and/or expandable tines provided at the distal end of the lead body 12. In some embodiments, the fixation member can be a screw-in fixation member. In other embodiments, the fixation member can be an extendable/retractable fixation member and can include one or more mechanical components adapted to facilitate the extension/retraction of the fixation member. An exemplary extendable/retractable fixation member is shown and described in U.S. Pat. No. 6,444,334 which is herein incorporated by reference.

The lead body 12 is flexible, but substantially non-compressible along its length and, in some embodiments, has a circular cross-section. Other lead body cross-sections can also be employed. According to one embodiment of the present invention, an outer diameter of the lead body 12 ranges from about 2 to about 15 French.

As shown in FIGS. 1B-1D, the lead body 12 is a multi-lumen lead body 12 and includes at least two lumens 38. In one embodiment, as shown in FIG. 1D, the lead body 12 includes four lumens 38. The lumens 38 can have a variety of cross-sectional shapes and can be of the same or different sizes. In one embodiment, at least one lumen 38 has an eccentric cross-sectional shape. The lumens 38 facilitate passage of the conductor from the connector 24 to the electrode and/or can receive a guiding element such as a guidewire or a stylet for delivery of the lead 10 to implant the lead 10 within a patient's heart.

The polymeric material used to form the lead body 12 can include a variety of different biocompatible polymeric materials, polymeric material blends, co-block polymers, co-polymers and elastomers used to manufacture lead bodies known to those of skill in the art. Exemplary polymeric materials include block co-polymer elastomers, polyurethane, polyurethane blends, polyurethane co-polymers, silicone rubbers, styrene-isobutylene-styrene (SIBS) co-polymers and the like. One example of a polyurethane co-polymer suitable for use in the present invention is a polyisobutylene urethane/urea co-polymer as shown and described in U.S. application Ser. No. 12/874,887, entitled “Medical Devices Including Polyisobutylene Based Polymers,” which is incorporated herein by reference in its entirety for all purposes.

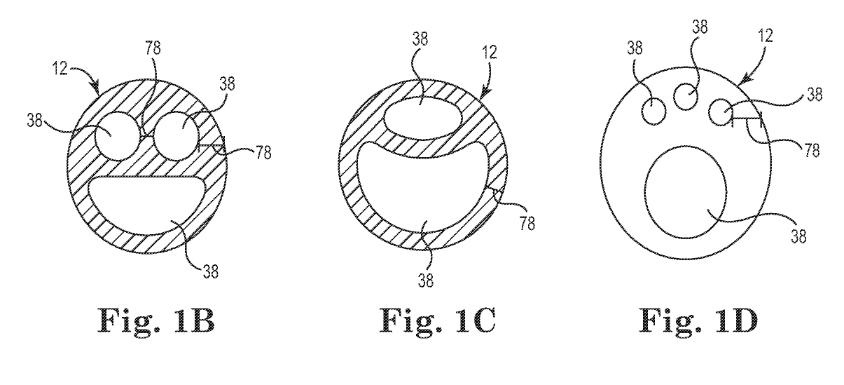

According to various embodiments of the present invention, as shown in FIG. 2, the lead body 12 is a multi-lumen lead body 12 and includes at least two portions, the approximate boundaries of which are illustrated by dashed lines. These portions can include a proximal portion 40, a middle portion 42, and a distal portion 44 including a lead tip portion 46. The proximal portion 40 generally represents portions of the lead body 12 that connect to the PG and/or lie subcutaneously in the patient's body. The middle portion 42 generally represents portions of the multi-lumen lead body 12 that reside in vessels that lead to the heart and/or in the upper chambers of the heart such as, for example, the right atrium. The distal portion 46 generally represents portions of the lead body 12 that reside within the heart, and generally includes at least one of the electrodes 28. In one embodiment, as shown in FIG. 2, the distal portion 46 also includes the pre-formed spiral fixation member 36. The lead tip portion 46 generally represents the distal end 20 of the lead body 12. The portions illustrated in FIG. 2 can vary in length and/or position on the multi-lumen lead body 12 depending on the type and size of the lead 10, the intended treatment and/or the intended implantation procedure.

According to some embodiments of the present invention, the durometer or stiffness of each of the different portions 40, 42, and 44 varies along the length of the multi-lumen lead body 12. In one embodiment, the stiffness of each of the portions 40, 42, and 44 decreases along the length of the body in a direction from the proximal end 16 to the distal end 20 of the multi-lumen lead body 12. For example, the proximal portion 40 has a stiffness that is greater than the stiffness of the distal portion 44, and the middle portion 42 has a stiffness that is less than the stiffness of the proximal portion 40 and greater than the stiffness of the distal portion 44. In one embodiment, the stiffness can decrease gradually and continuously from the proximal portion 40 to the tip portion 44 of the multi-lumen lead body 12. In other embodiments, the gradual decrease in stiffness of the lead body occurs in a stepwise manner. In one embodiment the stiffness of the material used to form the multi-lumen lead body 12 decreases from 75 D at the proximal portion of the lead body 12 to 45 A at the distal portion 44 including the tip portion 46 of the lead body. In further embodiments, the stiffness of the proximal portion ranges from about 85 A to about 100 D, the stiffness of the middle portion ranges from about 60 A to about 55 D and the stiffness of the distal portion ranges from about 30 A to about 70 A.

According to some embodiments, more than one polymeric material can be employed during the fabrication of the lead body 12. For example, in one embodiment, a polyurethane or a blend of polyurethanes and polyurethane co-polymers can be used to form a more proximal portion of the lead body 12 while silicone can be used to form a more distal portion of the lead body 12. In other embodiments, polyurethane co-polymers, silicone rubbers, styrene-isobutylene-styrene (SIBS) co-polymers, polyisobutylene based co-polymers, blends thereof and the like may be used.

The stiffness of the multi-lumen lead body 12 can be varied during the extrusion process used to form at least one portion of the lead body 12 such that at least one portion of the lead body 12 has a single unitary construction, and is not comprised of different segments that need to be bonded or otherwise joined together. For example, the durometer of the proximal portion 40 and/or middle portion 42 of the lead body 12 can be varied during the extrusion process. This can be accomplished using different extrusion techniques and methods, which are described according to the embodiments below.

In some embodiments, a mix or volume ratio of the polymeric material used to form at least one portion (proximal 40 and/or middle 42) of the multi-lumen lead body 12 can be varied during the extrusion process. In this embodiment, polymeric materials having different durometers are blended together and then extruded to form the different portions of the lead body 12. In one embodiment, a polymeric material available in different durometers can be blended together in different ratios of hard to soft polymeric material, and extruded to form the different portions of the lead body 12, described above. For example, a urethane having a Shore Hardness of about 80 A can be blended in different ratios with a urethane having a Shore hardness of about 50 A to form the at least one portion (proximal 40 and/or middle 42) of the lead body 12. In another embodiment, two or more different polymeric materials each having a different durometer can be blended together in different ratios of hard to soft and then extruded to form one or more portions of the lead body 12.

During the extrusion process, a volume percent of a stiffer polymeric material (Polymer A) in the polymeric material blend gradually changes from a maximum amount in the proximal portion 40 of the lead body 12 to a minimum amount in the middle portion 42 of the lead body 12 during extrusion of the lead body 12. Similarly, the volume percent of the softer polymeric material (Polymer B) in the polymeric blend changes from a minimum amount in the proximal portion 40 to a maximum amount in the middle portion of the lead body 12. In one embodiment, the durometer of Polymer A ranges from about 80 A to about 100 D, and the durometer of Polymer B ranges from about 25 A to about 40 A. In one embodiment, the volume ratio of Polymer A to Polymer B in the blend used to form proximal portion 40 of the lead body 12 ranges from about 75:25 to about 99:1. In one embodiment, the blend used to extrude the proximal portion 40 contains approximately 100% of Polymer A. In another embodiment, the volume ratio of Polymer A to Polymer B used to form a middle portion 42 of the lead body 12 ranges from about 35:65 to about 75:25.

In some embodiments, the distal portion and/or tip region of the multi-lumen lead body 12 can also be extruded and a mix or volume ratio of polymeric material used to form the distal and/or tip portions of the lead body 12 varied to vary the stiffness in the distal and/or tip portions 44, 46 of the lead body 12.

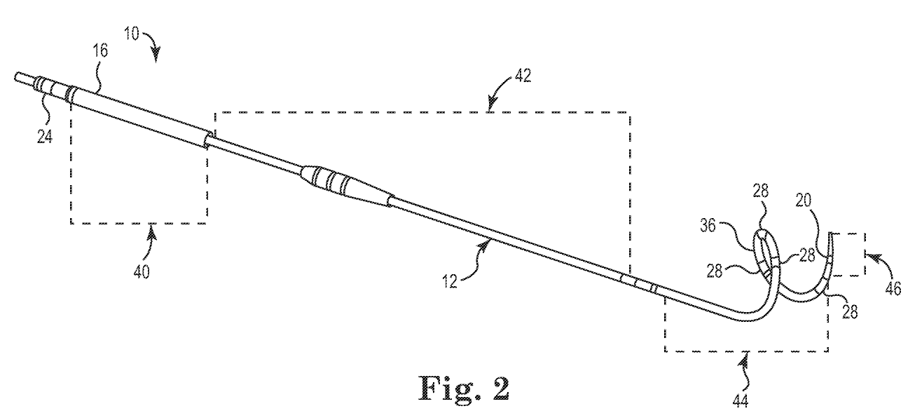

In other embodiments, at least one portion of the multi-lumen lead body 12 can be co-extruded to create a single, continuous lead body having an inner layer 52 and an outer layer 56 as shown in FIG. 3. For example, in one embodiment, the proximal portion 40 and/or middle portion 42 can be co-extruded. An exemplary co-extrusion method is generally shown and described in U.S. Pat. No. 6,135,922 entitled Medical Catheter and U.S. Pat. No. 5,542,937 entitled Multilumen Extruded Catheter, both of which are incorporated by reference herein in their entirety for all purposes.

In one embodiment, as depicted in FIG. 3, the cross-sectional wall thickness of each layer 52, 56 is varied along the length of the lead body 12 to vary the stiffness in the different portions (proximal 40 and/or middle 42) of the lead body 12. In one exemplary embodiment, the inner layer 52 can be extruded from a stiffer polymeric material (i.e. higher durometer polymer) and the outer layer 56 can be extruded from a softer polymeric material (i.e., lower durometer polymer). The inner layer wall thickness 62 transitions from a larger to a smaller percentage of the overall lead body wall thickness 64 along a length of the lead body in a direction towards the distal portion 44 of the lead body 12. Conversely, the outer layer wall thickness 66 transitions from a smaller percentage of the overall lead body wall thickness to a larger percentage of the overall lead body wall thickness along a length of the lead body 12 in a direction towards the distal portion 44 of the lead body 12. In one embodiment, the lead body 12 includes substantially the inner layer 52 at the proximal end 16 of the lead body 12 gradually transitioning to substantially the outer layer 56 in the middle portion 42 of the lead body 12. In another embodiment, the proximal portion 40 of the lead body 12 includes approximately 75-95% of the inner layer 52 having a higher durometer and approximately 5-25% of the outer layer 56 having a lower durometer. In one embodiment, as shown in FIG. 3, the overall outer diameter of the lead body 12 is maintained during the extrusion process. In another embodiment, the overall outer diameter of the lead body 12 is reduced as a result in the change in the thicknesses of each of the inner and outer layers 52, 56 in the different portions of the lead body 12. For example, in some embodiments, the proximal portion 40 of the lead body 12 can have a larger outer diameter than the middle portion 42 of the lead body 12.

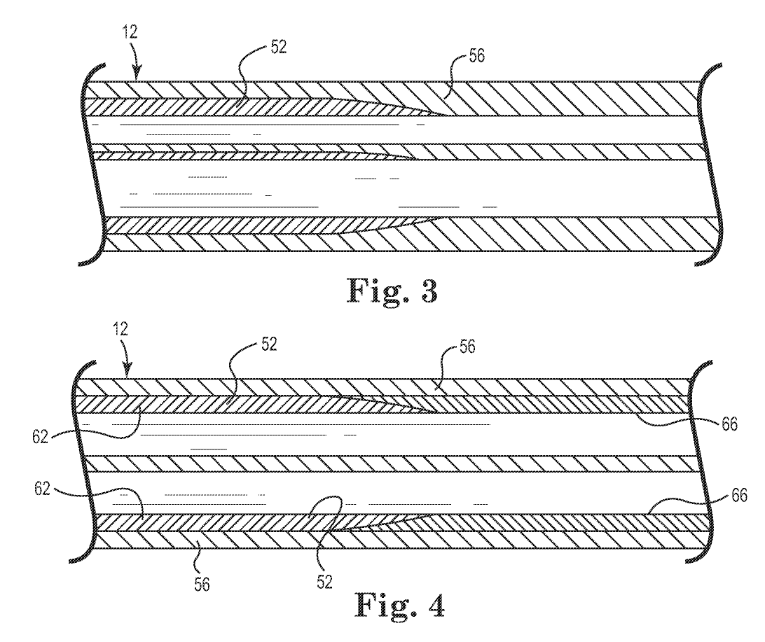

In addition to changing the cross-sectional wall thickness of each layer 52, 56 in some embodiments, the polymeric composition of one or both of the layers 52 or 56 can be changed during the extrusion process. For example, as shown in FIG. 4, the composition of the inner layer 52 can be changed from a first polymer 62 to a second polymer 66 during the extrusion process. This provides a further mechanism for manipulating the physical properties of the multi-lumen lead body 12 during the extrusion process to produce a multi-lumen lead body 12 having a variable stiffness along its length.

In another embodiment, the stiffness of at least one portion of the multi-lumen lead body 12 can be varied along its length by varying the inner geometry of the lead body 12 during the extrusion process. For example, the inner geometry of the proximal portion and/or middle portion 42 of the lead body 12 can be varied to vary the stiffness. In one embodiment, the inner geometry of the lead body 12 is varied without changing the overall outer dimensions of the lead body 12. Referring back to FIGS. 1B-1D, a wall thickness 78 of one or more lumens 38 can be varied along the length of at least one portion of the multi-lumen lead body 12 during the extrusion process to vary the stiffness of the lead body 12 in one or more selected portions (proximal 40 and/or middle 42). The wall thickness 78 is reduced by increasing an inner diameter and/or changing the cross-sectional shape of one or more of the lumens 38 extending within the multi-lumen lead body 12 during the extrusion process. According to various embodiments, a wall thickness 78 of one or more lumens 38 decreases in a direction from the proximal end 16 to the distal end 20 of the lead body 12. In one embodiment, a wall thickness 78 of one or more lumens 38 in a proximal portion 40 of the multi-lumen lead body 12 is greater than a wall thickness 78 of one or more lumens 38 in a middle region 42 of the multi-lumen lead body 12.

The inner wall thickness 78 can be altered by changing one or more extrusion parameters during the extrusion process. For example, in one embodiment, the draw down rate which is the extrusion rate versus material feed rate can be adjusted. In another embodiment, the air infiltration setting (which is a pressure setting) can be used to control the inner diameter of one or more select lumens 38. In yet another embodiment, an extruder apparatus having one or more mandrels or dies can be used to control and manipulate the inner diameter of one or more select lumens 38.

In one embodiment, an extruder apparatus having multiple mandrels of varying outer dimensions can be utilized to extrude a multi-lumen lead body 12. The internal dimensions of the different portions of multi-lumen lead body 12 (e.g. proximal 40, middle 42 and/or distal 44) are established by a mandrel over which the material to be extruded flows and the distal end of the mandrel is located at or near the die face where the material emerges from the extrusion head during the formation of the tubing, that is, the die face is in plane generally at a right angle to the flow of the material, or to the longitudinal axis of the mandrel, and is at the very end of the extrusion head. As such, the material flowing outwardly through the die face assumes the dimensions of the mandrel for its internal dimensions and the die for its external dimensions. Thus, the exterior dimensions of the mandrel can be varied by the user in accordance with the desired internal dimensions of the extruded multi-lumen lead body such that the user can predetermine those internal dimensions and still change such dimensions during the course of the extrusion process itself. An exemplary extrusion apparatus and method is generally shown and described in U.S. Pat. No. 6,926,509 entitled Apparatus for Extruding Tubing Having a Variable Wall Thickness, which is incorporated by reference herein in its entirety for all purposes.

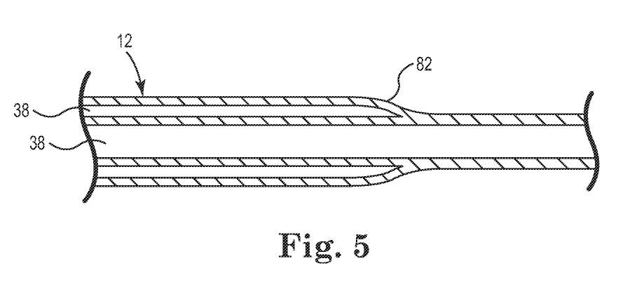

In some embodiments, the number of lumens in a select portion of the multi-lumen lead body 12 can be changed. In one embodiment, the number of lumens 38 in the lead body deceases along the length of the lead body in a direction towards the distal portion of the lead body 12. As a result of the change in the number of lumens, the overall outer diameter of the lead body 12 can be reduced resulting in an overall change in the durometer of the lead body 12 in that select portion. For example, FIG. 5 shows a portion of the lead body 12, according to one embodiment, in which the number of lumens 38 changes from two lumens 38 to a single lumen 38. In some embodiments, as shown in FIG. 5, the lead body 12 can include a neck-down region 82 where this transition occurs.

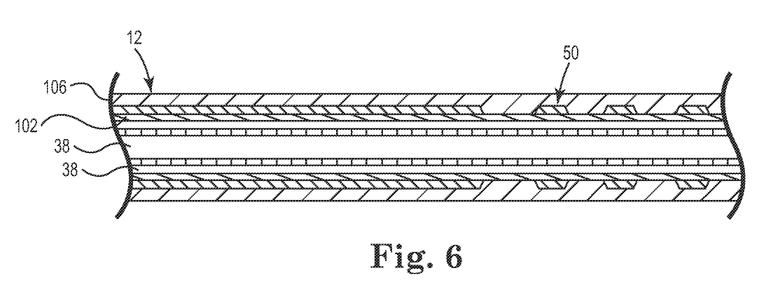

In yet another embodiment, a transition member 90 can be utilized to provide a smooth, continuous transition between portions of differing durometer on the multi-lumen lead body 12. In one embodiment, a transition member 90 is utilized to provide a smooth, continuous transition between the middle portion 42 and the distal portion 44 of the multi-lumen lead body 12. In a further embodiment, a transition member 90 is utilized to minimize the length of the transition between portions of the lead body 12 such as, for example, the middle portion 42 and the distal portion 44. FIG. 6 is cross-section view of a multi-lumen lead body 12 including a transition member 90 disposed between two material layers 102, 106 in a selected portion of the multi-lumen lead body. A transition member 90 can be used to provide a gradual, continuous transition in stiffness between the proximal portion 40 and the middle portion 42 and/or the middle portion 42 and the distal portion 44 of the lead body 12. In one embodiment, the distal portion 44 is molded over the transition member 90.

The transition member 90 maintains the axial load bearing capability between different portions of the lead body 12 while at the same time providing for flexibility of the lead body 12 during the transition from a higher durometer polymeric material to lower durometer polymeric material. The transition member 90 may also provide a mechanism for strengthening the mechanical interlocking of the insulative or non-insulative layers of material to the lead body 12 by increasing the amount of surface area available for adhesion. Increasing the mechanical boding strength may also facilitate strengthening the transition from a first polymeric material to a second polymeric material.

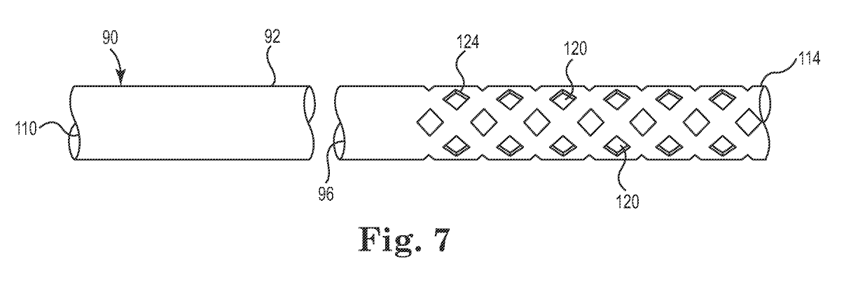

FIG. 7 is a schematic view of the transition member 90 according to an embodiment of the present invention. As shown in FIG. 7, the transition member 90 includes a tubular member 92 defining a lumen 96 extending between a proximal end 110 and a distal end 114. The tubular member 92 includes at least one recess 120 formed in an outer surface 124 of the tubular member 92.

The recesses 120 can result from mechanical, chemical, thermal or laser removal of material from the outer surface 124 of the tubular member 92. Thus, thinning of the tubular wall from a first region to a second region can also be considered a recess according to embodiments of the present invention. According to one embodiment of the present invention, the recesses 124 are depressions formed in the outer surface 124 of the tubular member 92. According to another embodiment, the recesses 120 are orifices that extend through the outer surface 124 of the tubular member 92 and into the lumen 96. The pattern, density, area, and location of the recesses 120 formed in the outer surface 124 can affect the flexibility of the reinforcing member 90, thus affecting the overall flexibility of a lead 10 when used in constructing the lead body 12. The pattern, density, area and location of the recesses 120 can be selected depending on the desired flexibility and other characteristics of the lead body 12 in a select region. According to one embodiment, the flexibility of the reinforcing member 90 increases in direct proportion to the number of recesses 120 formed in the outer surface 124 of the tubular member 92 and may provide a gradual transition from a less flexible region of the lead body 12 to a more flexible region of the lead body 12.

The tubular member 92 can be made from a polymeric material having a relatively higher durometer including polymers and polymer composites. In one embodiment the durometer of the material used to form the tubular member 92 ranges from about 70 A to about 100 A. The tubular member 92 should be fabricated from a material capable of withstanding the axial load requirements of the lead body even with one or more recesses 120 formed in an outer surface 124 of the tubular member 92. Exemplary materials include polycarbonates, polyacrylates, polyurethanes, polyesters, polyamides, polyethylenes, polypropylenes, polyvinylchloride, and polytetrafluoroethylene. According to one exemplary embodiment, the polymeric material used to fabricate the tubular member 92 is a polyurethane. Additionally, the wall thickness of the tubular member 92 should be sufficiently thin to achieve the flexibility desired between regions while at the same time maintaining the tensile strength and/or the tear resistance of the tubular member 92.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A medical electrical lead comprising:
a multi-lumen lead body having a proximal end, a distal end and at least two lumens extending within the lead body, the lead body including at least a first portion comprising a first polymeric material blend having a first ratio of at least two polymeric materials of differing durometers, the first portion gradually transitioning to a second portion located distal the first portion comprising a second polymeric material blend having a second ratio of the at least two polymeric materials of differing durometers and wherein the second polymeric blend has a lower durometer than the first polymeric material blend;
at least one conductor extending within one of the two or more lumens; and
at least one electrode located on the lead body and operatively connected to the at least one conductor.

2. The lead according to claim 1, wherein the first portion of the lead body comprises a blend of at least two polyurethane materials, each polyurethane material having a different durometer and the second portion comprises a silicone.

3. The lead according to claim 2, wherein a ratio of the at least two polyurethanes continuously changes along a longitudinal axis of the lead body in a direction from the proximal end towards the distal end of the lead body such that a stiffness of the lead body decreases in the same direction.

4. The lead according to claim 1, wherein the first portion of the lead body includes a first material layer having a first durometer and a second material layer having a second durometer, wherein the thickness of at least one of the layers is varied to vary a stiffness of the lead body such that the stiffness decreases along a longitudinal axis of the lead body in a direction from the proximal end towards the distal end of the lead body.

5. The lead according to claim 1, further comprising a tubular transition element disposed between and coupled to the first portion and the second portion, and wherein the second portion comprises silicone.

6. The lead according to claim 5, wherein the tubular transition element comprises a plurality of recesses, and wherein the second portion is molded over the transition element.

7. The lead according to claim 1, wherein a wall thickness of the first portion, second portion or both portions is decreased in a direction from a proximal end of the lead body toward the distal portion.

8. A medical electrical lead comprising:
a multi-lumen lead body having a proximal end, a distal end and at least two lumens extending within the lead body, the lead body including a proximal portion comprising a first material and a distal portion comprising a second material coupled to the proximal portion at a transition region; wherein a wall thickness of the proximal portion continuously decreases in a direction from the proximal end toward the distal portion;
at least one conductor extending within one of the two or more lumens; and
at least one electrode located on the distal portion of the lead body and operatively connected to the at least one conductor.

9. The lead according to claim 8, wherein an outer diameter of the proximal portion is reduced to reduce a stiffness of the proximal portion in a direction towards the distal portion.

10. The lead according to claim 8, wherein an inner geometry of at least one of the lumens is varied such that a stiffness of the proximal portion is reduced in a direction towards the distal portion.

11. The lead according to claim 8, wherein the transition region comprises a tubular transition element.

12. The lead according to claim 11, wherein the tubular transition element comprises a plurality of recesses, and wherein the distal portion is molded over the transition element.

13. A method of fabricating a multi-lumen lead body for a medical electrical lead including at least one portion having a varying durometer, the method comprising the steps of:
mixing a first polymeric material having a first durometer with a second polymeric material having a second durometer to form a mixture having a mix ratio of the first polymeric material to the second polymeric material; and
extruding the mixture to form at least one portion of the multi-lumen lead body while continuously altering the mix ratio to vary the stiffness of the at least one portion.

14. The method according to claim 13, wherein the mix ratio is altered such that the stiffness of the at least one portion is gradually reduced.

15. A method of fabricating a multi-lumen lead body for a medical electrical lead including at least one portion having a varying stiffness, the method comprising the steps of:
extruding at least a first polymeric material having a first durometer; and
continuously altering a wall thickness of the first material to vary the stiffness of the at least one portion of the lead body.

16. The method according to claim 15, further comprising extruding a second polymeric material having a second durometer over the first polymeric material and altering a wall thickness of the second material to vary the stiffness of the at least one portion of the lead body.

17. The method according to claim 15, further comprising maintaining an outer diameter of the lead body.

18. The method according to claim 15, further comprising reducing an outer diameter of the lead body.

19. The method according to claim 15, further comprising the step of mixing the first polymeric material with a second polymeric material having a second durometer to form a polymeric material blend having a mix ratio of the first material to the second material and altering the mix ratio of the first material to the second material to reduce the stiffness of the at least one portion during extrusion.

20. The method according to claim 15, further comprising the step of altering an inner geometry of at least one lumen to vary the stiffness of the at least one portion of the lead body.
